(19) United States
(12) Patent Application Publication
(10) Pub. No.: US 2021/0205378 A1
(43) Pub. Date: Jul. 8, 2021

(54) LACTIC ACID BACTERIA AND USE THEREOF

(71) Applicant: HOKUKON CO., LTD., Fukui (JP)

(72) Inventors: Yoshihito Tanaka, Fukui (JP); Minoru Kawagishi, Fukui (JP); Masaru Sato, Fukui (JP); Saki Fujita, Fukui (JP)

(21) Appl. No.: 17/058,826

(22) PCT Filed: Apr. 1, 2019
(86) PCT No.: PCT/JP2019/014496

§ 371 Date: Nov. 25, 2020

(30) Foreign Application Priority Data
May 31, 2018 (JP) 2018-104321

(51) Int. Cl. A61K 35/744; A01N 63/20; A23K 10/16; A23L 33/135; C04B 28/02; C05F 11/08; C12N 1/20

(52) U.S. Cl. CPC A61K 35/744; A01N 63/20; A23K 10/16; C12N 1/20; C04B 28/02; C05F 11/08; A23L 33/135

(57) ABSTRACT

A lactic acid bacterial strain Enterococcus durans (E. durans) HS-08 or a processed product thereof is provided having IgA production promoting effects, immunostimulation, and antiallergic effects. The product also provides an inhibitory effect on proliferation of harmful bacteria and/or pathogenic bacteria, protective effect on the mucosa, enhancing effect on short chain fatty acid production, enhancing effect on organic acid production, upregulating effect on the expression of the GRP43 gene, modulating effect on plant growth, preventive effect on plant lodging, adjustment effect on the umami taste of plants, promoting effect on animal growth, or enhancement of immunity in animals.

### TECHNICAL FIELD

The present invention relates to a novel lactic acid bacterium or a processed product thereof, a method for increasing the number of the lactic acid bacterium or the amount of the processed product, and use of the bacterium or the processed product.

### BACKGROUND ART

Lactic acid bacteria are widely used in fermented foods such as yogurt and pickles, and are very familiar to people and very safe. Some lactic acid bacteria have recently attracted attention as probiotics, i.e., microorganisms that confer health benefits on humans. Studies have been advanced to examine various efficacy of such lactic acid bacteria.

Lactic acid bacteria have various functions. For example, lactic acid bacteria that induce IgA production have previously been reported (Patent Literatures 1 and 2). Lactic acid bacteria and a processed product thereof have also been reported to induce IgA production. Lactic acid bacteria and a processed product thereof have the potential to prevent infectious diseases, inhibit tumors, inhibit allergies, promote gastrointestinal health, and have other effects. Lactic acid bacteria and a processed product thereof are also known to be applicable as an immunostimulator, an intestinal health promoter, or other agents (Patent Literature 3). However, the effects of lactic acid bacteria and a processed product thereof may greatly vary depending on the bacterial strains, even when the bacterial strains belong to the same bacterial species. Selection of a desirable bacterial strain is therefore very important, and many attempts have been made to isolate a lactic acid bacterial strain with high functions.

### CITATION LIST

### Patent Literature

Patent Literature 1: JP 2004-305128 A
Patent Literature 2: JP 2010-130954 A
Patent Literature 3: JP 2008-201708 A

### SUMMARY OF INVENTION

### Technical Problem

An object of the present invention is to provide a novel lactic acid bacterial strain with high functions or a processed product thereof. Another object of the present invention is to provide a method for producing the lactic acid bacterial strain or a processed product thereof, or use of the lactic acid bacterial strain or a processed product thereof.

### Solution to Problem

The inventors conducted extensive research to solve the above problems, and successfully isolated the lactic acid bacterial strain Enterococcus durans (hereinafter also referred to as “E. durans”) HS-08 (Accession No. NITE BP-02675) and found that this strain has many great utilities. The inventors made further study based on this finding and completed the present invention.

The present invention was made to solve the above problems and includes the following.

(1) A Enterococcus durans HS-08 bacterium (Accession No. NITE BP-02675) or a processed product thereof.
(2) A composition for induction of IgA production or for immunostimulation, the composition comprising the bacterium or a processed product thereof according to the above (1).
(3) An antiallergic composition comprising the bacterium or a processed product thereof according to the above (1).
(4) A composition for inhibition of proliferation of harmful bacteria and/or pathogenic bacteria, the composition comprising the bacterium or a processed product thereof according to the above (1).
(5) A composition for protection of mucosa, the composition comprising the bacterium or a processed product thereof according to the above (1).
(6) A composition for at least one application selected from the group consisting of enhancement of production of short chain fatty acids, enhancement of production of organic acids, and upregulation of gene expression of GRP43, the composition comprising the bacterium or a processed product thereof according to the above (1).
(7) A composition for at least one application selected from the group consisting of modulation of plant growth, prevention of plant lodging, and adjustment of umami taste of a plant, the composition comprising the bacterium or a processed product thereof according to the above (1).
(8) A composition for promotion of growth of an animal and/or for enhancement of immunity in an animal, the composition comprising the bacterium or a processed product thereof according to the above (1).
(9) A fertilizer for plants, comprising the bacterium or a processed product thereof according to the above (1) or the composition according to the above (7).
(10) A feed for animals, comprising the bacterium or a processed product thereof according to the above (1) or the composition according to the above (8).
(11) A food or drink product, a cosmetic product or a pharmaceutical product, comprising the bacterium or a processed product thereof according to the above (1) or the composition according to any one of the above (2) to (8).
(12) A concrete member comprising the bacterium or a processed product thereof according to the above (1) or the composition according to any one of the above (2) to (8).
(13) A composition comprising the bacterium or a processed product thereof according to the above (1) and at least one selected from the group consisting of beneficial bacteria including other lactic acid bacteria and bifidobacteria, beneficial yeasts, and molds, or a processed product thereof.
(14) A method for increasing the number of Enterococcus durans HS-08 bacteria, the method comprising culturing Enterococcus durans HS-08 bacteria (Accession No. NITE BP-02675).

### Advantageous Effects of Invention

The present invention provides the lactic acid bacterial strain Enterococcus durans (hereinafter also referred to as “E. durans”) HS-08 (Accession No. NITE BP-02675) (hereinafter also referred to as the “lactic acid bacteria of the present invention”) or a processed product thereof.

The lactic acid bacteria of the present invention significantly induce the production of the immune antibody IgA, thereby promoting immunostimulation in a living body.

The lactic acid bacteria of the present invention also have antiallergic effect. Surprisingly, the lactic acid bacteria also have inhibitory effect on proliferation of harmful bacteria and/or pathogenic bacteria, protective effect on the mucosa, enhancing effect on short chain fatty acid production, enhancing effect on organic acid production, upregulating effect on the expression of the GRP43 gene, modulating effect on plant growth, preventive effect on plant lodging, adjustment effect on the umami taste of plants, promoting effect on animal growth, or immunity enhancing effect.

### BRIEF DESCRIPTION OF DRAWINGS

FIG. 1 is a chart showing the IgA levels (μg/day) in the feces of mice.

FIG. 2 is a chart showing the IgA concentration (μg/mL) in the blood of mice.

FIG. 3 is a chart showing the body weight (g) of mice.

FIG. 4 is a chart showing the CRE concentration (μg/mL) in the blood of mice.

FIG. 5 is a chart showing the ALT concentration (iU/L) in the blood of mice.

FIG. 6 is a chart showing the AST concentration (iU/L) in the blood of mice.

FIG. 7 is a chart showing the expression levels of various genes involved in IgA production.

FIG. 8 is a chart showing organic acid concentrations (μmol/g) and short chain fatty acid concentrations (μmol/g) in the feces of mice.

FIG. 9 is a chart showing the pH of the feces of mice.

FIG. 10 is a chart showing the expression level of the GRP43 gene.

### DESCRIPTION OF EMBODIMENTS

The E. durans HS-08 strain (Accession No. NITE BP-02675) was deposited with Patent Microorganisms Depositary (NPMD) of Biological Resource Center in Incorporated Administrative Agency National Institute of Technology and Evaluation (address: 2-5-8 Kazusakamatari, Kisarazu-shi, Chiba 292-0818, Japan) on Apr. 10, 2018, as an international deposit under the Budapest Treaty.

The bacteria can be obtained from the depository center by submitting the request. Alternatively, the bacteria may be those that are identified by comparison of the characteristics with those of the E. durans HS-08 strain (Accession No. NITE BP-02675), and the characteristics include, for example, morphological characteristics (for example, the shape of colonies, the shape of cells, etc.), physiological or biochemical characteristics (for example, utilization of sugars, growth temperature, optimal pH, etc.), and chemotaxonomic characteristics (fatty acid composition of bacterial cells, etc.). The bacteria may be those that are identified based on nucleotide sequence analysis of 16S rRNA genes.

The E. durans HS-08 strain is preferably cultured, for example, as follows.

(i) Culture broth (culture medium) includes MRS (de Man, Rogosa and Sharpe) medium, MRS agar medium, LBS medium, modified LBS agar medium, Rogosa medium, or other known media or broth for lactic acid bacteria.
(ii) The pH is about 4 to 9, preferably about 5 to 8, more preferably about 5.5 to 7.5.
(iii) The bacteria may be cultured by static culture or spinner culture. The frequency of rotation of the spinner in spinner culture may be about 100 to 250 revolutions per minute, is preferably about 120 to 180 revolutions per minute, and is more preferably about 140 to 160 revolutions per minute.
(iv) Duration of culture is typically about 1 to 72 hours, preferably about 3 to 48 hours, more preferably about 6 to 24 hours.
(v) Culture temperature is typically about 20 to 40° C., preferably about 25 to 39° C., more preferably about 30 to 38° C.
(vi) Supplements may be added to MRS medium, MRS agar medium, LBS medium, modified LBS agar medium, Rogosa medium, and other known media or broth for lactic acid bacteria.

Examples of components or supplements added to the above media include, but are not limited to, yeast-derived components, soybean-derived components, corn-derived components; animal and plant proteins or extracts and decomposed products thereof; ammonium salts, such as ammonium nitrate, ammonium sulfate, ammonium chloride, and ammonium acetate; one or more of nitrogen sources selected from the group consisting of ammonia, sodium nitrate, potassium nitrate, sodium glutamate, urea, amino acids, gluten, casein, peptone, etc.; and one or more of carbon sources selected from the group consisting of glucose, inositol, maltose, xylose, mannose, fructose, starch, lactose, glycerol, arabinose, ribose, galactose, fructose, inositol, mannitol, sorbitol, glucosamine, cellobiose, sucrose, trehalose, xylitol, alcohols, starch syrup, starch, molasses, glycerin, organic acids, organic acid salts, and hydrocarbons. Examples of the components or supplements also include, but are not limited to, vitamins, such as vitamins A, B, C, D, E, and K, and derivatives thereof, and salts thereof; minerals, such as zinc, iron, magnesium, potassium, calcium, and phosphorus; pH buffering agents; surfactants; antibiotics; and stabilizers. Other examples of the components or supplements include, but are not limited to, sucrose, carbonates, hydrogencarbonates, HEPES, albumin, insulin, amino acids, cytokines, growth factors, and hormones. These supplements can be used alone or in combination of two or more types. The anaerobic or aerobic culture conditions may be established according to a procedure known in the art.

The lactic acid bacteria of the present invention may be formed into a powder by lyophilization, low-temperature drying, spray drying or L-drying, or a combination thereof. Alternatively, the lactic acid bacteria prepared as above may be used as it is (in the form of a paste, a liquid, etc.). When the lactic acid bacteria are used in a powder form, the number of viable bacteria is preferably, but not limited to, 107 to 10⁸ cfu/g, more preferably 109 to 10¹² cfu/g. When the lactic acid bacteria are used in a liquid form, the number of viable bacteria is preferably, but not limited to, 107 to 10⁸ cfu/mL, more preferably 109 to 10¹² cfu/mL. The method for quantifying the number of viable bacteria may be selected depending on the types of bacterial cells, and the number of viable bacteria may be easily determined by, for example, the quantification method for various bacterial cells as described in the Japanese Pharmaceutical Codex.

The “processed product” of the lactic acid bacteria of the present invention or other lactic acid bacteria is preferably, but not limited to, a processed or cultured product of the lactic acid bacteria. The lactic acid bacteria may be viable bacteria or dead bacteria. The processed product may be used as it is, or may be formed into a powder by lyophilization, low-temperature drying, spray drying, L-drying, or a combination thereof according to the present invention. The processed product (cultured product) may be diluted in an appropriate solvent (water, an alcohol, an organic solvent, etc.), or may be formed into a gel or a solid preparation by addition of an appropriate additive.

The lactic acid bacteria or a processed product thereof according to the present invention may be used in combination with other beneficial bacteria (for example, lactic acid bacteria including those belonging to the genus Lactobacillus, Leuconostoc, or Streptococcus; bifidobacteria; yeasts; molds etc.) or a processed product thereof. Examples of said other lactic acid bacteria include, for example, but are not limited to, the genus Lactobacillus, such as Lactobacillus acidophilus, Lactobacillus delbrueckii, Lactobacillus casei, Lactobacillus leichmannii, Lactobacillus fructivorans, Lactobacillus helveticus, Lactobacillus hilgardii, Lactobacillus kefiri, Lactobacillus kefiranofaciens, Lactobacillus kefirgranum, Lactobacillus parakefir, Lactobacillus pentoaceticus, Lactobacillus caret, Lactobacillus bulgaricus, Lactobacillus lactis, Lactobacillus plantarum, Lactobacillus paracasei, Lactobacillus cellobiosus, Lactobacillus confusus, Lactobacillus coprophilus, Lactobacillus fermentum, Lactobacillus sanfrancisco, Lactobacillus thermophilus, Lactobacillus bavaricus, Lactobacillus brevis, Lactobacillus buchneri, Lactobacillus coryniformis, Lactobacillus curvatus and Lactobacillus sake; the genus Leuconostoc, such as Leuconostoc cremoris and Leuconostoc mesenteroides; and the genus Streptococcus, such as Streptococcus lactis, Streptococcus salivarius and Streptococcus thermophilus.

These beneficial bacteria can be cultured by a conventionally known method, and are easily available from organizations such as ATCC (registered trademark) or IFO; Japan Bifidus Foundation; or Independent administrative institution National Institute of Technology and Evaluation, Patent Microorganisms Depositary. Commercially available bacteria may also be used as appropriate.

Examples of bifidobacteria that can be used in combination with the lactic acid bacteria or a processed product thereof according to the present invention include, but are not limited to, for example, the genus Bifidobacterium or its subspecies, such as Bifidobacterium bifidum, Bifidobacterium angulatum, Bifidobacterium adolescentis, Bifidobacterium breve, Bifidobacterium catenulatum, Bifidobacterium dentiurn, Bifidobacterium gallicum, Bifidobacterium subtile, Bifidobacterium asteroides, Bifidobacterium boum, Bifidobacterium choerinum, Bifidobacterium coryneforme, Bifidobacterium cuniculi, Bifidobacterium gallinarum, Bifidobacterium indicum, Bifidobacterium magnum, Bifidobacterium merycicum, Bifidobacterium minimum, Bifidobacterium psychraerophilum, Bifidobacterium pullorum, Bifidobacterium ruminantium, Bifidobacterium scardovii, Bifidobacterium thermophilurn, Bifidobacterium infantis, Bifidobacterium mongoliense, Bifidobacterium pseudocatenulatum, Bifidobacterium thermacidophilum subsp. porcinum, Bifidobacterium animalis subsp. lactis, Bifidobacterium animalis subsp. animalis, Bifidobacterium longum subsp. longum, Bifidobacterium pseudolongum subsp. pseudolongum and Bifidobacterium pseudolongum subsp. globosum. These bifidobacteria can be cultured by a conventionally known method, and are easily available from various organizations or depositaries, as exemplified for the above lactic acid bacteria. Commercially available bacteria may also be used as appropriate.

Examples of yeasts that can be used in combination with the lactic acid bacteria or a processed product thereof according to the present invention include, but are not limited to, the genus Saccharomyces, such as Saccharomyces cervisiae (also known as budding yeast or top fermentation yeast), Saccharomyces kefir, Saccharomyces lactis, Saccharomyces carlsbergensis (also known as bottom fermentation yeast), Saccharomyces unisporus, Saccharomyces pastrianus, Saccharomyces bayanus, Saccharomyces carlsbergensis, Saccharomyces delbrueckii, Saccharomyces dairensis, Saccharomyces diastaticus, Saccharomyces exiguus, Saccharomyces kluyveri, Saccharomyces rouxii, Saccharomyces uvarum and Saccharomyces rosei; the genus Schizosaccharomyces, such as Schizosaccharomyces pombe (also known as fission yeast) and Schizosaccharomyces rouxii; the genus Candida, such as Candida utilis (also known as Torula yeast), Candida tropicalis, Candida milleri, Candida krusei, Candida lusitaniae, Candida aaseri, Candida guilliermondii, Candida humicola, Candida glabrata, Candida lambica, Candida lipolytica, Candida parapsilosis, Candida tropicalis, Candida paratropicalis, Candida pseudotropicalis, Candida rugosa, Candida stellatoidea and Candida zeylanoides; the genus Torulaspora, such as Torulaspora delbrueckii; the genus Torulopsis, such as Torulopsis celluculosa and Torulopsis candida; the genus Torula, such as Torula kefir; the genus Kluyveromyces, such as Kluyveromyces bulgaricus, Kluyveromyces fragilis, Kluyveromyces thermotolerans, Kluyveromyces lactis, Kluyveromyces marxianus and Kluyveromyces fragilis; the genus Pichia, such as Pichia membranaefaciens, Pichia stipitis, Pichia anomala and Pichia saitoi; the genus Hansenula, such as Hansenula anomala; and the genus Debaryomyces, such as Debaryomyces hansenii. These yeasts can be cultured by a conventionally known method, and are easily available from various organizations or depositaries, as exemplified for the above lactic acid bacteria. Commercially available bacteria may also be used as appropriate.

Examples of other beneficial bacteria that can be used in combination with the lactic acid bacteria or a processed product thereof according to the present invention include, but are not limited to, acetic acid bacteria including the genus Acetobacter, such as Acetobacter aceti, Acetobacter orientalis, Acetobacter pasteurianus and Acetobacter xylinum; butyric acid bacteria including Clostridium butyricum; the genus Pediococcus, such as Pediococcus damnosus, Pediococcus pentosaceus and Pediococcus halophilus; filamentous fungi (aspergillus); Bacillus subtilis, such as Bacillus subtilis natto; Actinomyces; photosynthetic bacteria; amino acid-producing bacteria; cellulose-decomposing bacteria (e.g., Clostridium bacteria, Trichoderma bacteria, etc.); root nodule bacteria; mycorrhizal fungi; VA fungi; and nitrifying bacteria. These yeasts can be cultured by a conventionally known method, and are easily available from various organizations or depositaries, as exemplified for the above lactic acid bacteria. Commercially available bacteria may also be used as appropriate.

Examples of molds that can be used in combination with the lactic acid bacteria or a processed product thereof according to the present invention include, but are not limited to, the genus Aspergillus, such as Aspergillus oryzae, Aspergillus sojae, Aspergillus mucor, Aspergillus niger, Aspergillus flavus and Aspergillus nidulans; the genus Penicillium, such as Penicillium roqueforti and Penicillium rhizopus. These molds can be cultured by a conventionally known method, and are easily available from various organizations or depositaries, as exemplified for the above lactic acid bacteria. Commercially available bacteria may also be used as appropriate.

The lactic acid bacteria or a processed product thereof according to the present invention or a composition comprising the lactic acid bacteria or a processed product thereof may be directly sprayed onto (administered to) plants (leaves, stems, fruits thereof, etc.) or soil. The lactic acid bacteria or a processed product thereof or the composition may also be added to fertilizers for plants, or may be placed on sluice gates etc. in farm fields or rice fields, or may be added to the materials of concrete for producing sluice gates or other concrete structures. When the lactic acid bacteria or a processed product thereof according to the present invention or the composition comprising the lactic acid bacteria or a processed product thereof is used in the livestock industry, the lactic acid bacteria or a processed product thereof or the composition may be added to feeds or drinking water and administered to animals, or may be added to materials for making concrete for concrete structures, such as walls, floors, etc. of cattle barns, poultry houses, etc.

The present invention provides a composition comprising the lactic acid bacteria of the present invention. The composition of the present invention may further contain a known additive typically used in the art, for example, including, but not limited to, water, solvents, pH adjusting agents, moisturizers, flavoring agents, sweeteners, thickeners, flavor improvers, gelling agents, solubilizers, colorants, antiseptics, surfactants, suspending agents, emulsifiers and stabilizers.

The pH adjusting agents that may be used in the present invention may be, for example, any pH adjusting agent capable of adjusting the pH of the composition of the present invention to a desired pH level. Such a pH adjusting agent can be used to adjust the pH of the composition to about 2.5 to 10.0, preferably about 5.0 to 8.5. Examples of preferred pH adjusting agents include alkali metal hydroxides, alkaline earth metal hydroxides, alkaline earth metal oxides, alkali metal oxides, carbonic acid salts, boric acid salts, silicic acid salts, phosphoric acid salts, organic acids, and organic bases.

Specific examples of the pH adjusting agents include, but are not limited to, phosphoric acid or salts thereof; benzoic acid or salts thereof; salicylic acid or salts thereof; sodium hydroxide; potassium hydroxide; alkali metal carbonates, such as sodium carbonate, potassium carbonate, and magnesium carbonate; alkaline earth metal carbonates, such as magnesium hydroxide and calcium hydroxide; alkali metal bicarbonates, such as potassium hydrogen carbonate and sodium hydrogen carbonate; alkaline earth metal bicarbonates, such as magnesium hydrogen carbonate and calcium hydrogen carbonate; boric acid, citric acid, fumaric acid salts, malic acid, tartaric acid, succinic acid, maleic acid, and salts thereof; and lactic acid, imidazole, triethanolamine, diethanolamine, trometamol, meglumine, lidocaine, and salts thereof.

Examples of preferred moisturizers that can be used in the present invention include, but are not limited to, polyhydric alcohols, such as sorbitol, xylitol, glycerin, butylene glycol, polyethylene glycol, and propylene glycol.

Examples of the flavoring agents that can be used in the present invention include, but are not limited to, peppermint oil, spearmint oil, mentha oil, menthol, anethole, sage, lemon oil, orange oil, cinnamon, vanillin, thymol, and linalool.

Examples of the sweeteners that can be used in the present invention include, but are not limited to, sucrose, glucose, saccharin, glycyrrhizic acid, dextrose, fruit sugar, lactose, mannitol, sorbitol, fructose, maltose, xylitol, honey, starch syrup, saccharin, aspartame, D-tryptophan, acesulfame, cyclamic acid, and salts thereof.

Examples of the flavor improvers that can be used in the present invention include, but are not limited to, ascorbic acid, citric acid, glycyrrhizic acid, glutamic acid, succinic acid, tartaric acid, fumaric acid, malic acid, taurine, sarcosine, glycyrrhizic acid, and salts thereof; and erythritol, lactitol, reduced palatinose, sodium chloride, magnesium chloride, orange oil, saffronoil, Japanese pepper oil, perilla oil, basil oil, mentha oil, lemon oil, lemongrass oil, rose oil, rosemary oil, cacao, caramel, liquorice, camphor, cinnamon oil, saffron, methyl salicylate, peony extract, ginger, cinnamaldehyde, stevia extract, swertia herb, sorbitol, cyclodextrin, soybean oil, Ziziphus jujuba fruit extract, taurine, tannicacid, clove oil, bitter orange peel extract, Picrasma quassioides extract, Japanese apricot extract, honey, mentha water, mentha oil, menthol, povidone, borneol, eucalyptus oil, lemon oil, and rose oil.

The surfactants that can be used in the present invention may be nonionic, zwitterionic, anionic, or cationic surfactants. The nonionic surfactants may be any suitable one and examples thereof include, but are not limited to, polyoxyethylene sorbitan fatty acid esters, such as polysorbate 20, polysorbate 40, polysorbate 60, and polysorbate 80; polyoxyethylene hydrogenated castor oils, such as polyoxyethylene hydrogenated castor oil 10, polyoxyethylene hydrogenated castor oil 40, polyoxyethylene hydrogenated castor oil 50, and polyoxyethylene hydrogenated castor oil 60; polyoxyethylene polyoxypropylene glycol; and polyethylene glycol fatty acid esters, such as polyethylene glycol monolaurate, polyethylene glycol monostearate (such as ethylene glycol monostearate and polyoxyl 40 stearate), polyethylene glycol monooleate, and ethylene glycol monostearate. Examples of the anionic surfactants include, but are not limited to, sodium alkylbenzene sulfonate and ammonium dodecylbenzenesulfonate. Examples of the zwitterionic surfactants include, but are not limited to, aliphatic derivatives of secondary or tertiary amines or of heterocyclic secondary or tertiary amines, which derivatives contain a carboxy, sulfonate, or sulfate group; for example, (C₈₋₂₀ alkyl) betaines, (C₈₋₂₀ alkyl) amide (C₆₋₈ alkyl)betaines, and mixtures thereof. Examples of the cationic surfactants include, but are not limited to, quaternary ammonium salts, amine salts, and amines.

Examples of preferred emulsifiers and suspending agents that can be used in the present invention include the surfactants as exemplified above. Other examples of preferred emulsifiers and suspending agents include, but are not limited to, lecithins, such as soybean lecithin, yolk lecithin, hydrogenated lecithin, and enzymatically decomposed lecithin; and higher alcohols, such as cetanol, lauryl alcohol, stearyl alcohol, and lanolin alcohol.

Examples of the stabilizers that can be used in the present invention include, but are not limited to, sodium polyacrylate, adipic acid, ascorbic acid, sodium sulfite, sodium hydrogen sulfite, dibutylhydroxytoluene, butylated hydroxyanisole, sodium edetate, sodium chloride, citric acid, cyclodextrin, and cysteine.

The lactic acid bacteria or a processed product thereof according to the present invention, or the composition comprising the lactic acid bacteria or a processed product thereof may be administered to animals including humans. The lactic acid bacteria or a processed product thereof according to the present invention or the composition may be formulated into feeds (agricultural materials), pharmaceuticals or food products or mixed with the ingredients of feeds (agricultural materials), pharmaceuticals or food products and administered to animals. Alternatively, the lactic acid bacteria or a processed product thereof according to the present invention or the composition may be formulated into pharmaceuticals or cosmetic products or mixed with the ingredients of pharmaceuticals or cosmetic products and applied to animals.

The animals to which the composition of the present invention may be administered may be any types of animals, including, but not limited to, for example, humans and non-human animals. Examples of the non-human animals include, but are not limited to, livestock animals, such as cattle, horses, pigs, and sheep; pets or experimental animals, such as dogs, cats, rabbits, hamsters, squirrels, guinea pigs, and mice; fishes, such as sardine, tuna, sharks, sunfish, rays, horse mackerel, stringfish, yellowtail, sweetfish, carp, sea bream, mackerel, olive flounder, righteye flounder, eel, moray eel, goosefish, pufferfish, thread-sail filefish, and tropical fishes; shellfishes, such as decapods, crabs, squids, scallops, oysters, and turban shells; birds, such as chickens, quails, turkeys, domestic ducks, geese, parakeet, and parrots; insects, such as beetles, stag beetles, cabbage butterflies, swallowtail butterflies, mantises, grasshoppers, bell crickets, and pillbugs; reptiles, such as geckos, chameleons, iguanas, and lizards; and amphibians, such as newts, frogs, and salamanders. Especially preferred are humans, cattle, pigs, chickens, fishes, and decapods.

The mode of administration used in the present invention may be any mode of administration, including oral administration, parenteral administration (intravenous administration, transdermal administration, etc.), etc. The dosage form used in the present invention may be an oral formulation, including tablets, capsules, granules, and powders, or a parenteral formulation, including injections, ointments, and insert dosage forms.

When the dosage form used in the present invention is, for example, a tablet, a capsule, a granule, a powder, or the like, such a dosage form can be formulated using, as needed, one or more additional ingredients selected as appropriate from, but not limited to, for example, fillers, such as mannitol, calcium phosphate, starch, saccharose, lactose, and glucose; disintegrants, such as carboxymethyl cellulose, starch, pregelatinized starch, calcium carboxymethylcellulose, croscarmellose sodium, crospovidone, and low-substituted hydroxypropyl cellulose; binders, such as hydroxypropyl cellulose, ethyl cellulose, gum arabic, starch, partially pregelatinized starch, polyvinylpyrrolidone, and polyvinyl alcohol; lubricants, such as magnesium stearate, calcium stearate, talc, hydrous silicon dioxide, and hydrogenated oils; and coating agents, such as polyvinylpyrrolidone, sugars, hydroxypropyl methylcellulose, hydroxypropyl cellulose, methyl cellulose, and ethyl cellulose.

When the formulation of the present invention is intravenously administered, for example, in an injectable dosage form, such a dosage form can be formulated using, as needed, one or more additional ingredients selected as appropriate from, but not limited to, for example, isotonic agents, such as sodium chloride; buffering agents, such as sodium phosphate; surfactants, such as polyoxyethylene sorbitan monooleate; and thickeners, such as methyl cellulose.

When the formulation of the present invention is locally administered to the eye, for example, in an eye drop dosage form etc., such a dosage form can be formulated using, as needed, one or more additional ingredients selected as appropriate from, but not limited to, for example, isotonic agents, such as sodium chloride and concentrated glycerin; buffering agents, such as sodium phosphate and sodium acetate; surfactants, such as polyoxyethylene sorbitan monooleate, polyoxyl 40 stearate, and polyoxyethylene hydrogenated castor oil; stabilizers, such as sodium citrate and sodium edetate; and antiseptics, such as benzalkonium chloride and paraben. The formulation may be at any pH that is acceptable for ophthalmic formulations, and the pH may be adjusted using a pH adjusting agent, for example, as described later.

When the formulation of the present invention is in the form of an ointment, a transdermal agent, a patch, or the like, such a dosage form can be formulated using a commonly used base, for example, a higher fatty acid, such as petrolatum, squalane, paraffin, liquid paraffin, lauric acid, myristic acid, stearic acid, isostearic acid, and oleic acid; or a fat and fatty oil, such as waxes, including beeswax and lanolin.

When the formulation of the present invention is in the form of an insert dosage form, such a dosage form can be formulated using a biodegradable polymer, such as hydroxypropylcellulose, hydroxypropyl methylcellulose, carboxyvinyl polymer, and polyacrylic acid. If necessary, another additional ingredient selected from the stabilizers, the pH adjusting agents, etc. as exemplified above can also be used as appropriate.

The dosage according to the present invention is not limited to a particular one and may be selected as appropriate depending on the dosage form, the symptoms, age and body weight of a patient, etc. For example, for oral administration, 0.05 to 5000 mg per kg body weight per day, preferably 0.1 to 2000 mg per kg body weight per day, particularly preferably 1 to 1000 mg per kg body weight per day, can be administered in a single dose or several divided doses a day. For an injection, 0.0001 to 2000 mg per kg body weight per day, preferably 0.001 to 1500 mg per kg body weight per day, particularly preferably 0.01 to 500 mg per kg body weight per day, can be administered in a single dose or several divided doses a day.

### Food or Drink Products and Food Additives

The present invention provides a food product comprising the lactic acid bacteria or a processed product thereof according to the present invention or a composition comprising the lactic acid bacteria or a processed product thereof. The food product includes health foods, foods with functional claims, foods for specified health use, and foods for sick people. The food product may be in any form. Specific examples of the food product include, but are not limited to, dietary supplements, supplements in the form of tablets, granules, powders, energy drinks, etc. Other examples of the food product include drinks such as refreshing drinks, nutritional drinks, carbonated drinks, fruit juice, lactic drinks and tea drinks; noodles such as buckwheat noodles, wheat noodles, Chinese noodles, and instant noodles; sweets such as chocolate, snacks, candies, chewing gum, biscuits, jelly, jam, and cream; bakery products; fishery and livestock products, such as sausages, fish sausages, and hams; dairy products such as processed milk and fermented milk (including yogurt); fats, oils and processed foods thereof, such as margarine, mayonnaise, shortening, whipped cream, dressing, vegetable oil, and oil for deep frying; seasonings such as soy sauce and sauce; retort pouch foods such as curry, rice-bowl cuisines, porridge, rice soup, and stew; and frozen desserts, such as ice cream, and sherbet. The amount of the E. durans HS-08 strain contained in the food product is preferably 0.1 to 10%, more preferably 1 to 10%, of the total mass of the food product.

The present invention provides a food additive comprising the lactic acid bacteria or a processed product thereof according to the present invention or a composition comprising the lactic acid bacteria or a processed product thereof. Examples of the food additive include, but are not limited to, for example, pH adjusting agents, preservatives, antimicrobial agents, antioxidants, antifungal agents, colorants, color fixatives, bleaching agents, brightening agents, fragrances, spice extracts, softening agents, nutrition fortifiers, sweeteners, acidulants, seasonings, bittering agents, emulsifiers, thickeners, stabilizers, gelling agents, thickening agents, inflating agents, processing aids, enzymes, gum bases, yeast foods, and formulations of these food additives. The additives etc. as described above can also be used. The amount of the E. durans HS-08 strain contained in the food additive is preferably 0.1 to 10%, more preferably 1 to 10%, of the total mass of the food additive.

### Cosmetic Products

The present invention provides a cosmetic product comprising the lactic acid bacteria or a processed product thereof according to the present invention or a composition comprising the lactic acid bacteria or a processed product thereof. The cosmetic product includes the so-called medicated cosmetics (quasi drugs). Examples of the cosmetic product include, but are not limited to, for example, washing lotions, shampoos, rinses, hair tonics, hair lotions, aftershave lotions, body lotions, makeup lotions, cleansing creams, massage creams, emollient creams, aerosol products, deodorizers, aromas, deodorants, and bath fragrances. Depending on the purpose, the cosmetic product of the present invention may further contain, as appropriate, a component generally used in cosmetic products in addition to the lactic acid bacteria or a processed product thereof or the composition of the present invention, and such a component includes, for example, surfactants, moisturizers, fats and oils from animals and plants, fats and oils from microorganisms, silicones, higher alcohols, lower alcohols, extracts from animals and plants, extracts from microorganisms, ultraviolet absorbers, anti-inflammatories, sequestering agents, vitamins, antioxidants, thickeners, preservatives, bactericides, pH adjusting agents, colorants, and various fragrances. Specific examples of these components include those exemplified above. The amount of the E. durans HS-08 strain contained in the cosmetic product is preferably 0.1 to 10%, more preferably 1 to 10%, of the total mass of the cosmetic product.

### Pharmaceutical Products

The present invention provides a pharmaceutical product comprising the lactic acid bacteria or a processed product thereof according to the present invention or a composition comprising the lactic acid bacteria or a processed product thereof. The pharmaceutical product contains an active ingredient in addition to the lactic acid bacteria of the present invention, and can be formulated with addition of a pharmaceutically acceptable carrier and/or additive if necessary, as appropriate. Specific examples of the pharmaceutical product include, but are not limited to, tablets, coated tablets, pills, powders, granules, capsules, solutions, suspensions, emulsions, injections, infusions, suppositories, ointments, sprays, salves, creams, gels, and patches. The blending ratio of a carrier or additive is determined as appropriate based on the range usually employed in the pharmaceutical field. The carrier or additive that can be combined is not limited to a particular one, and examples thereof include various types of carriers, such as water, physiological saline, other aqueous solvents, aqueous or oily bases; and various types of additives such as excipients, binders, pH adjusting agents, disintegrants, absorption enhancers, lubricants, colorants, flavor improvers and fragrances. In addition to these, those as exemplified above can also be used as appropriate. The amount of the E. durans HS-08 strain contained in the pharmaceutical product is preferably 0.1 to 10%, more preferably 1 to 10%, of the total mass of the pharmaceutical product.

### Animal Feeds

The present invention provides a feed for animals, comprising the lactic acid bacteria or a processed product thereof according to the present invention or a composition comprising the lactic acid bacteria or a processed product thereof. Examples of the animal feed include, but are not limited to, feeds for livestock animals, such as cattle, horses, pigs, and sheep; feeds for pets or experimental animals, such as dogs, cats, rabbits, hamsters, squirrels, guinea pigs, and mice; feeds for fishes, such as sardine, tuna, sharks, sunfish, rays, horse mackerel, stringfish, yellowtail, sweetfish, carp, sea bream, mackerel, olive flounder, righteye flounder, eel, moray eel, goosefish, pufferfish, thread-sail filefish, and tropical fishes; feeds for shellfishes, such as decapods, crabs, squids, scallops, oysters, and turban shells; feeds for birds, such as chickens, quails, turkeys, domestic ducks, geese, parakeet, and parrots; feeds for insects, such as beetles, stag beetles, cabbage butterflies, swallowtail butterflies, mantises, grasshoppers, bell crickets, and pillbugs; feeds for reptiles, such as geckos, chameleons, iguanas, and lizards; and feeds for amphibians, such as newts, frogs, and salamanders. The animal feed of the present invention can be produced by adding the lactic acid bacteria of the present invention to a feed in accordance with a conventional production method for animal feeds. The amount of the E. durans HS-08 strain contained in the animal feed is preferably 0.1 to 10% by mass, more preferably 1 to 10% by mass, of the total mass of the animal feed. The animal feed, when given to animals (in particular, livestock animals or poultry), promotes the growth of the animals, enhances immunity of the animals, shortens the feeding period of the animals, or shortens the days until the shipment of the animals.

### Plant Fertilizers

The present invention provides a fertilizer for plants, comprising the lactic acid bacteria or a processed product thereof according to the present invention or a composition comprising the lactic acid bacteria or a processed product thereof. The fertilizer may be a fertilizer for plants, including, but not limited to, vegetables, flowers, flowering trees and shrubs, and fruits. Specific examples of the vegetables and fruits include root crops, such as Japanese radish, burdock, carrots, turnips, and lotus root; tuber crops, such as potatoes, sweet potatoes, Chinese yams, taros, Japanese taros, konjac yams, and Japanese yams; beans, such as green soybeans, broad beans, peas, kidney beans, lentils, adzuki beans, and sesame seeds; leaf vegetables, such as cabbage, lettuce, Chinese cabbage, spinach, nozawana, potherb mustard, Japanese honewort, Japanese mustard spinach, and chrysanthemum greens; citruses, such as mandarin orange, amanatsu orange, pomelos, ponkan orange, iyokan orange, hassaku orange, lemons, limes, and grapefruits; allium vegetables, such as onions, Japanese green onion, and Japanese leek; mushrooms, such as cucumbers, golden needle mushroom, king trumpet mushroom, wood ear mushroom, shimeji mushroom, nameko mushroom, matsutake mushroom, shiitake mushroom, and maitake mushroom; flower vegetables, such as broccoli, cauliflower, canola flower, and artichoke; gourds, such as cucumbers, watermelons, Japanese pumpkin, zucchini, calabashes, sponge gourd, winter melon, squirting cucumber, white-flowered gourd, bitter melon, balsam pear, and melons; grain cereals, such as paddy rice, rye, adlay millet, proso millet, foxtail millet, Japanese barnyard millet, and sorghum; other grain cereals, such as wheat and barley; edible wild plants, such as bracken ferns and Japanese royal ferns; cycad; ginkgo; pine; bamboo; peaches; chestnuts; persimmons; strawberries; apples; pears; grapes; muscat; tomatoes; eggplants; Japanese horseradish; celery; bamboo shoots; and garlic. Examples of the flowers and flowering trees and shrubs include, but are not limited to, flowers, such as thistle, iris, baby's breath, wood sorrel, dandelion, pea flower, garden balsam, carnation, chamomile, primrose, lily, salvia, peony, canola flower, chrysanthemum, buttercup, red spider lily, pansy, violet, sunflower, hyacinth, tulip, narcissus, cotton rosemallow, sweet pea, marigold, lily of the valley, dianthus pink, gerbera, geranium, zinnia, balloon flower, cosmos, dahlia, morning glory, bindweed, crocus, cockscomb, and hydrangea; and flowering trees and shrubs, such as the family Rosaceae including rose, Japanese apricot, cherry, peach, Japanese quince, Japanese plum, and apricot; the family Oleaceae including fragrant olive, jasmine, holly osmanthus, lilac, forsythia, and winter jasmine; the family Cornaceae including flowering dogwood; the family Thymelaeaceae; the family Fabaceae including wisteria and bush clover; the family Theaceae including common camellia and sasanqua camellia; the family Ericaceae including satsuki azalea, azalea, rhododendron, Japanese andromeda, andblueberry; and the family Malvaceae; the family Aceraceae; the family Thymelaeaceae; the family Styracaceae; the family Hippocrateaceae; the family Anacardiaceae; and the family Caprifoliaceae. The fertilizer of the present invention can be produced by adding the lactic acid bacteria of the present invention to a fertilizer in accordance with a conventional production method for fertilizers. The amount of the E. durans HS-08 strain contained in the fertilizer is preferably 0.1 to 10% by mass, more preferably 1 to 10% by mass, of the total mass of the fertilizer. The fertilizer for plants may have modulating effect on plant growth, preventive effect on plant lodging, adjustment effect on the umami taste of plants, or other effects. The fertilizer for plants may promote the growth of, for example, the roots, stems, branches, leaves, fruits or flowers of plants. The fertilizer for plants may also enhance the umami taste of, for example, the roots, stems, branches, leaves, fruits or flowers of plants.

### Industrial Products

The present invention provides an industrial product (e.g., a concrete member etc.) comprising the lactic acid bacteria or a processed product thereof according to the present invention or a composition comprising the lactic acid bacteria or a processed product thereof. Examples of industrial products other than concrete members include, but are not limited to, for example, petroleum products, resin products, magnetic products, leather articles, and woven fabrics. The amount of the E. durans HS-08 strain contained in the industrial product is preferably 0.1 to 10% by mass, more preferably 1 to 10% by mass, of the total mass of the industrial product.

The industrial product comprises the lactic acid bacteria or a processed product thereof according the present invention, which is effective for promotion of animal growth, enhancement of immunity in animals, modulation of plant growth, prevention of plant lodging, adjustment of the umami taste of plants, or others, or comprises the composition comprising the lactic acid bacteria or a processed product. Therefore, when the industrial product is in the form of products used for raising animals (e.g., feedboxes, fences, etc.) or products used for growing plants (e.g., planters, watering pots, buckets, hoses, a concrete member of sluice gates, etc.), the industrial product is useful for promotion of animal growth, enhancement of immunity in animals, modulation of plant growth, prevention of plant lodging, adjustment of the umami taste of plants, or others.

### Kit

The present invention provides a kit comprising the lactic acid bacteria or a processed product thereof according to the present invention or a composition comprising the lactic acid bacteria or a processed product thereof. The kit preferably contains a container capable of separately storing individual dosage forms of the composition, for example, separated bottles or foil packets. Alternatively, individual dosage forms of the composition can also be stored in a single container. Examples of preferred forms of the kit include, but are not limited to, blister packs for packaging tablets, capsules or the like, and syringes or containers that are filled with a drug solution. The kit may be sterilized by a known technique, such as irradiation and autoclave.

### Immunostimulatory Effect

The lactic acid bacteria of the present invention preferably have immunostimulatory effect. For example, when a group with administration of the lactic acid bacteria of the present invention or a composition comprising the lactic acid bacteria shows or tends to show a significantly higher IgA concentration in the feces or blood than that of a non-administration group, the lactic acid bacteria or the composition may be determined to have immunostimulatory effect. For example, the composition of the present invention, when added to a feed and administered to animals, alleviates the symptoms of immune diseases (e.g., mastitis, colibacillosis, diarrhea, etc.). Due to this effect, the amount of antibiotics used can also be reduced.

Infections with pathogenic bacteria or viruses occur when these bacteria or viruses attach to the surface of epithelial cells. IgA produced from the Peyer's patch prevents such attachment of bacteria or viruses, and thus plays an important role in the immune system. The Peyer's patch is present in the intestinal tract, and is composed of T cells, B cells, and plasma cells, which produce IgA, and other cells.

Other substances are also known to have immunostimulatory effect on mucous tissue. Acetic acid induces the production of retinoic acid, which induces the production of IgA. IL-6, RALDH2, APRIL, BAFF, etc. secreted from dendritic cells in the Peyer's patch also induce IgA production. Therefore, for example, when a group with administration of the lactic acid bacteria of the present invention or a composition comprising the lactic acid bacteria shows or tends to show a significantly higher acetic acid concentration or significantly higher expression of the RALDH2, IL-6, APRIL and BAFF genes than those of a non-administration group, the lactic acid bacteria or the composition may be determined to stimulate the immunity of mucous tissue, thereby preventing infections. The mucous tissue is preferably, for example, those found in the digestive organs, respiratory organs, urinary organs, etc. More preferably, the lactic acid bacteria or the composition stimulates the immune system of mucous tissue in the intestinal tract.

### Protective Effect on Mucosa

The lactic acid bacteria of the present invention preferably have protective effect on the mucosa. Organic acids such as lactic acid and acetic acid produced by enterobacteria contribute to various biological responses of animals including humans. Such organic acids are absorbed by mucous tissue, and used as the energy source for mucosal epithelium cells to repair epithelial cells and to protect the mucosa, as previously described. Therefore, for example, when a group with administration of the lactic acid bacteria of the present invention or a composition comprising the lactic acid bacteria shows or tends to show a significantly higher amount of lactic acid and/or acetic acid in mucous tissue than that of a non-administration group, the lactic acid bacteria or the composition may be determined to have protective effect on the mucosa.

### Antiallergic Effect

The lactic acid bacteria of the present invention preferably have antiallergic effect. For example, when a group with administration of a composition comprising the lactic acid bacteria of the present invention shows or tends to show a significantly higher IgA concentration in the blood or feces than that of a non-administration group, the composition may be determined to have antiallergic effect. For example, the composition of the present invention, when administered to humans, alleviates the symptoms of pollen allergy or other allergic disorders.

### Enhancing Effect on Production of Short Chain Fatty Acids and Organic Acids

The lactic acid bacteria of the present invention preferably have enhancing effect on the production of short chain fatty acids and organic acids. Short chain fatty acids or organic acids produced by enterobacteria such as lactic acid bacteria contribute IgA production, as previously described. Therefore, for example, when a group with administration of the lactic acid bacteria of the present invention or a composition comprising the lactic acid bacteria shows or tends to show significantly higher concentrations of short chain fatty acids or organic acids than those of a non-administration group, the lactic acid bacteria or the composition may be determined to have effect on the production of short chain fatty acids or organic acids.

Examples of the short chain fatty acids or organic acids of which the production is enhanced by the lactic acid bacteria of the present invention include, but are not limited to, lactic acid and acetic acid. Short chain fatty acids are known to exhibit immunostimulatory effect and antiallergic effect via induction of IgA production, and may provide various beneficial effects on the body of humans or animals.

### Upregulating Effect on Gene Expression of GRP43

The lactic acid bacteria of the present invention preferably have upregulating effect on the gene expression of the acetic acid receptor GRP43. GRP43 is the receptor for short chain fatty acids and has a particularly high affinity against acetic acid. GRP43 is upregulated with an increase in the amount of short chain fatty acids in the intestines, as previously described. In other words, upregulation of the expression of the GRP43 gene may induce an increase in short chain fatty acids (such as acetic acid) in the intestinal tract.

Upregulation of the expression of the GPR43 gene provides various useful effects, including prevention of diabetes mellitus via the modulation of insulin signaling, and suppression of colitis via induction of regulatory T cells, which are involved in maintenance of homeostasis (Nature Communications volume 4, Article number: 1829 (2013)). Therefore, for example, when a group with administration of the lactic acid bacteria of the present invention or a composition comprising the lactic acid bacteria shows or tends to show significantly higher expression of the GRP43 gene than that of a non-administration group, the lactic acid bacteria or the composition may be determined to have preventive effect on diabetes mellitus and colitis.

Inhibitory Effect on Proliferation of Harmful Bacteria and/or Pathogenic Bacteria

The lactic acid bacteria of the present invention preferably have inhibitory effect on proliferation of harmful bacteria and/or pathogenic bacteria. Proliferation of bacteria that are harmful or pathogenic to animals, such as humans and mice, is inhibited in acidic environment, as previously described. Thus an increase in organic acids, such as lactic acid and acetic acid, acidifies the intestinal environment and reduces the pH, and may inhibit the proliferation of harmful or pathogenic bacteria in the intestines. For example, when a group with administration of the lactic acid bacteria of the present invention or a composition comprising the lactic acid bacteria shows or tends to show a significantly lower pH level in the intestines than that of a non-administration group, the lactic acid bacteria or the composition may be determined to have inhibitory effect on proliferation of harmful bacteria and/or pathogenic bacteria.

The harmful bacteria and pathogenic bacteria whose proliferation may be inhibited by the present invention include, but are not limited to, Clostridium perfringens, Escherichia coli, etc.

### Modulating Effect on Plant Growth, Preventive Effect on Plant Lodging and Adjustment Effect on Umami Taste of Plants

The lactic acid bacteria of the present invention preferably have modulating effect on plant growth. For example, when a group with administration of the lactic acid bacteria of the present invention or a composition comprising the lactic acid bacteria shows or tends to show significant growth in the size of the roots, leaves, branches, fruits or flowers than that of a non-administration group, for example, when the size of the roots, leaves, branches, fruits or flowers of the administration group grows by preferably about 10 to 100%, more preferably about 30 to 50%, as compared with that of a non-administration group, the lactic acid bacteria or the composition may be determined to have modulating effect on plant growth. Due to this effect, the amount of fertilizers used for plant growth can also be reduced.

The lactic acid bacteria of the present invention preferably have preventive effect on plant lodging. The degree of plant lodging can be expressed in terms of, for example, the degree of inclination of lodging plants in a six-step scale ranging from no inclination (0) to very strong inclination (5). For example, the lodging rate of a group with administration of the lactic acid bacteria of the present invention or a composition comprising the lactic acid bacteria can be reduced by preferably 1 to 5 scale points, more preferably 2 to 4 scale points, as compared with that of a non-administration group, but the degree of reduction is not limited to the above scale points. The plants may be, for example, those exemplified above, and are preferably, but not limited to, rice or paddy rice. The composition may further contain a mineral, such as calcium, zinc, iron, magnesium, potassium and phosphorus. The composition, when further contains a mineral, has further enhanced preventive effect on plant lodging. This effect may be due to enhancement of the immunity of plants by the composition, and due to this effect, the amount of fertilizers used can also be reduced.

The lactic acid bacteria of the present invention preferably have adjustment effect on the umami taste (sugar content) of plants. For example, the sugar content of plants may be increased by preferably about 1% to about 70%, more preferably about 20% to about 50%, as compared with that before the administration of the lactic acid bacteria, but the degree of an increase in the sugar content is not limited thereto. The plants may be, for example, those exemplified above, and are preferably, but not limited to, rice, melon, mandarin orange, lemon, tomato, Chinese cabbage, etc.

### Promoting Effect on Animal Growth and Enhancing Effect on Immunity in Animals

The lactic acid bacteria of the present invention preferably have promoting effect on animal growth. For example, the body weight of a group with administration of the lactic acid bacteria of the present invention or a composition comprising the lactic acid bacteria can be increased by about 10 to 200% by mass, more preferably about 30 to 100% by mass, as compared with that of a non-administration group, or the body length of a group with administration of the lactic acid bacteria of the present invention or a composition comprising the lactic acid bacteria can be increased by about 10 to 200%, more preferably about 30 to 100%, as compared with that of a non-administration group, but the degree of increase is not limited thereto. Due to this effect, the amount of feeds used can also be reduced. The animals may be, but are not limited to, for example, those exemplified above.

The lactic acid bacteria of the present invention preferably have enhancing effect on the immunity in animals. For example, when a group with administration of the lactic acid bacteria of the present invention or a composition comprising the lactic acid bacteria shows or tends to show a significantly higher IgA concentration in the feces or blood than that of a non-administration group, the lactic acid bacteria or the composition may be determined to have enhancing effect on the immunity. For example, the composition of the present invention, when added to a feed and administered to animals, alleviates the symptoms of immune diseases (e.g., pollen allergy, allergy, etc.). The animals may be, but are not limited to, for example, those exemplified above.

### EXAMPLES

The present invention will be described in more detail below with reference to Examples, but is not limited thereto.

### Example 1: IgA Production-Inducing Activity of the E. durans HS-08 Strain

(1) Male BALB/cA mice were purchased at 6 weeks of age and subjected to the tests described below. Immediately after arrival, the mice were given an acclimation period of 17 days to avoid stress from the new environment, which might affect the test results. During the acclimation, the mice were fed with a purified feed only (AIN-76A, Research Diets, New Brunswick, N.J.). After the acclimation period, the feeds described below were administered to the mice, and the mice were observed for a predetermined period (about 60 days).
(2) The mice after acclimation were divided into four groups of approximately equal body weights and IgA levels in the feces. Two test classes were created, one with no administration of the E. durans HS-08 strain (hereinafter referred to as the control group), and the other with administration of the E. durans HS-08 strain at 0.38% in the feed (hereinafter referred to as the 0.38% group). The number of animals in each test class was five (Table 1).

TABLE 1

| Class | Number of animals | Percentage of E. durans in feed |
|---|---|---|
| Control group | 5 | 0% |
| 0.38% group | 5 | 0.38% |

(3) A lactic acid bacteria powder for adding to the feed for mice was prepared by culturing the E. durans HS-08 strain in MRS medium (Difco Laboratories, Detroit, Mich., USA). Specifically, a colony of the E. durans HS-08 strain was first inoculated in 5 mL of MRS broth, and statically cultured under sealed conditions at 37° C. for 24 hours. The cultured bacteria in the broth were then placed into 2000 mL of fresh MRS broth and statically cultured under sealed conditions at 37° C. for 24 hours. The culture broth was centrifuged to collect the bacteria, and a 10% sucrose aqueous solution as a vehicle was added in a 2-fold volume of the bacterial cells. The bacterial suspension was lyophilized to prepare a lactic acid bacteria powder according to the present invention. The number of viable bacteria contained in the lactic acid bacteria powder was 1.1×10¹² cfu/g.
(4) The formulations of the feeds for the test classes are shown in Table 2.

TABLE 2

| Formulation | Control group | 0.38% group |
|---|---|---|
| Purified feed (%, w/w) | 100.00 | 99.300 |
| Lactic acid bacteria powder (%, w/w) | 0.00 | 0.70 (0.38) |
| (the value in the bracket indicates the | | |
| percentage of E. durans in feed) | | |

(5) The feces of mice excreted per day were collected on days 0, 14, 28, 42 and 60 after the start of feeding with the lactic acid bacteria powder. The feces were mixed with phosphate buffered saline containing a protease inhibitor and placed on ice for 30 minutes. The liquid mixture was centrifuged. The IgA concentration in the supernatant was determined using Mouse IgA ELISA kit.
(6) The blood was withdrawn from the mice on day 60 after the start of feeding with the lactic acid bacteria powder. The blood was mixed with heparin sodium and centrifuged. The IgA concentration in the supernatant (hereinafter also referred to as the “plasma obtained in Example 1”) was determined using Mouse IgA ELISA kit.

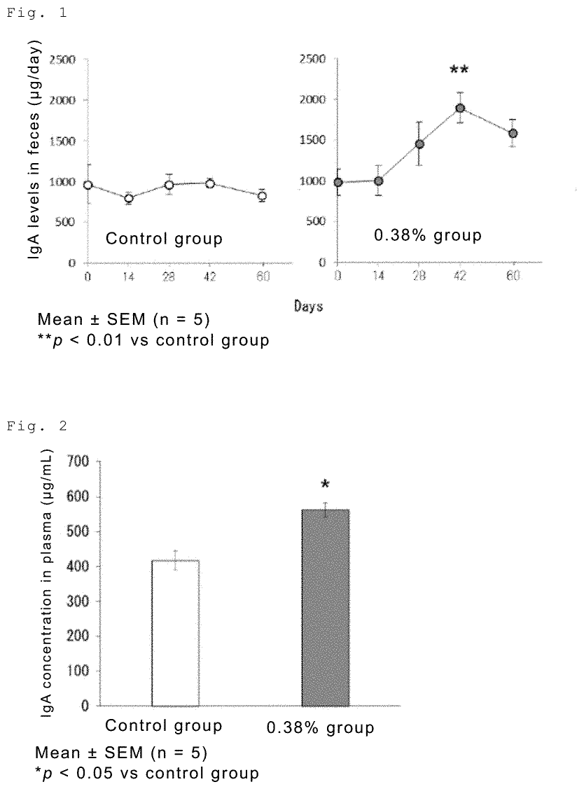

FIG. 1 shows the time course of the IgA levels. As shown in the figure, the 0.38% group showed the tendency to an increase in the IgA levels on day 28 as compared with the control group, and showed a significant increase in the IgA levels on day 42. The IgA levels decreased on day 60 as compared with those on day 42, but still tended to be higher than those of the control group. The data of the IgA levels in the feces were statistically analyzed by two-way repeated measures ANOVA, followed by multiple comparison test. Other data were statistically analyzed by multiple comparison test.

As apparent from Table 3 and FIG. 2, the IgA concentration in the blood of the 0.38% group was significantly higher than that of the control group.

TABLE 3

| Class | IgA concentration in blood (μg/ml) |
|---|---|
| Control group | 417.1 ± 28.6 |
| 0.38% group | 561.2 ± 20.2* |

Mean ± SEM (n = 5)
*p < 0.05 vs control group

The group fed with the lactic acid bacteria powder (0.38% group) showed higher IgA levels in the feces than those of the control group. These results indicate that the E. durans HS-08 strain stimulates the intestinal immune system.

The group fed with the lactic acid bacteria powder (0.38% group) also showed a higher IgA concentration in the blood than that of the control group. Increase in the IgA concentration in the blood indicates enhancement of systemic immune activity, and hence confirmed that the E. durans HS-08 strain stimulates the systemic immune system.

### Example 2: Safety Test of E. durans HS-08 Strain

(1) After the acclimation period, the body weight was measured on days 0, 7, 14, 21, 28, 35, 42, 49, 56 and 60 after the start of feeding with the lactic acid bacteria powder.
(2) The concentration of creatinine (CRE) in the plasma obtained in Example 1 was determined using LabAssay Creatinine. The creatinine (CRE) concentration in the blood increases when there are abnormalities of the kidney. The purpose of this test was to compare the CRE concentration between the control group and the group fed with the lactic acid bacteria powder to assess the safety of the lactic acid bacteria powder on the kidney function of mice.
(3) The concentrations of alanine aminotransferase (ALT) and aspartate aminotransferase (AST) in the plasma obtained in Example 1 were determined using Transaminase CII kit. The ALT and AST concentrations in the blood increase when there are abnormalities of the liver. The purpose of this test was to compare the ALT and AST concentrations between the control group and the group fed with the lactic acid bacteria powder to assess the safety of the lactic acid bacteria powder on the liver function of mice.
(4) The concentration of IgE in the plasma obtained in Example 1 was determined using Mouse IgE ELISA kit. The purpose of this test was to compare the IgE concentration between the control group and the group fed with the lactic acid bacteria powder to ensure that lactic acid bacteria do not cause any allergic reaction in mice.

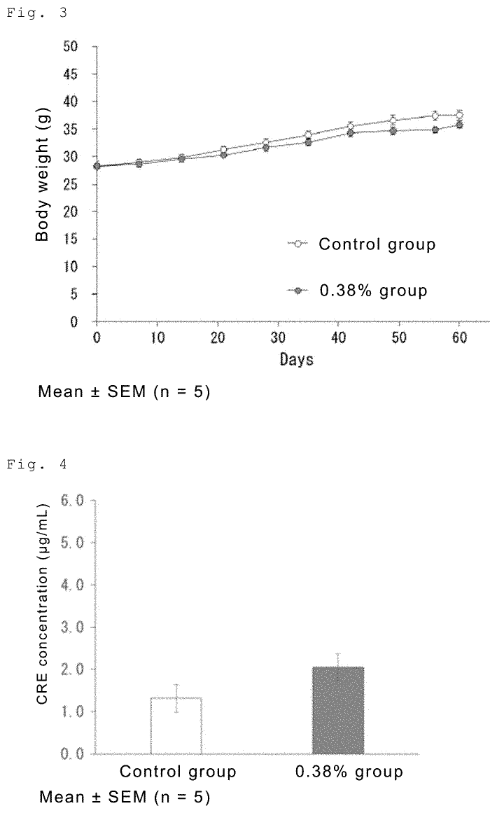

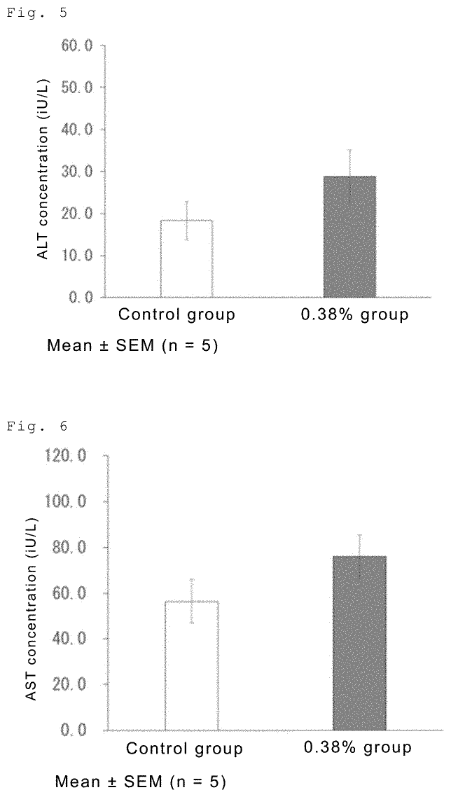

Comparison of the body weight showed no significant difference between the control group and the group fed with the lactic acid bacteria powder (FIG. 3). Comparison of the CRE concentration also showed no significant difference between the control group and the group fed with the lactic acid bacteria powder (Table 4 and FIG. 4). Comparison of the ALT and AST concentrations also showed no significant difference between the control group and the group fed with the lactic acid bacteria powder (Table 5 and FIGS. 5 and 6). IgE was not detected in either the control group or the group fed with the lactic acid bacteria powder.

TABLE 4

| Class | CRE concentration (μg/ml) |
|---|---|
| Control group | 1.3 ± 0.3 |
| 0.38% group | 2.0 ± 0.3 |

Mean ± SEM (n = 5)

TABLE 5

| Class | ALT concentration (iU/l) | AST concentration (iU/l) |
|---|---|---|
| Control group | 18.3 ± 4.6 | 56.3 ± 9.6 |
| 0.38% group | 28.9 ± 6.2 | 76.0 ± 9.5 |

Mean ± SEM (n = 5)

The body weight and the CRE, ALT and AST concentrations of the group fed with the lactic acid bacteria powder showed no significant difference as compared with the control group. These results indicate that ingestion of the E. durans HS-08 strain does not appear to adversely affect the body weight, the kidney and the liver functions of mice.

IgE was not detected in either the control group or the group fed with the lactic acid bacteria powder. These results indicate that the E. durans HS-08 strain also does not appear to cause allergic reaction to mice, i.e., is not an allergen to mice.

Drug resistance test, the Limulus test and other tests were also conducted, demonstrating the safety of the lactic acid bacteria of the present invention. The results are tabulated in Table 6 below.

TABLE 6

| | | Lactic acid bacteria |
|---|---|---|
| | Control group | 0.38% group |
| Feed intake (g d⁻¹) | 4.2 ± 0.3 | 4.4 ± 0.2 |
| Initial body weight (g) | 28.0 ± 0.2 | 28.4 ± 0.7 |
| Final body weight (g) | 37.5 ± 0.8 | 35.8 ± 0.6 |
| Body weight gain (g) | 9.5 ± 0.7 | 7.4 ± 0.4 |
| Blood chemistry profile | | |
| AST (iU l⁻¹) | 56.3 ± 9.6 | 76.0 ± 9.5 |
| ALT (iU l⁻¹) | 18.3 ± 4.6 | 28.9 ± 6.2 |
| CRE (μg ml⁻¹) | 1.3 ± 0.3 | 2.0 ± 0.3 |
| IgA (μg ml⁻¹) | 417.1 ± 28.6 | 561.2 ± 20.2 |

Mean ± SEM (n = 5)

### Example 3: Gene Analysis of Peyer's Patch Cells

The mice prepared in accordance with the procedures (1) to (4) in Example 1 were fed with the lactic acid bacteria powder. The mice were euthanized by cervical dislocation on day 60 after the start of feeding with the lactic acid bacteria powder, and the Peyer's patch cells were collected from the mice. RNA was extracted using illustra RNAspin Mini RNA Isolation Kit (GE Healthcare, Piscataway, N.J., USA). A 0.15 μg sample of the RNA was weighed and reverse transcribed with oligo primers (Invitrogen, Carlsbad, Calif.) using SuperScript III Reverse Transcriptase (Invitrogen). The reverse transcribed sample was then purified using PCR Purification Kit (Qiagen, Cambridge, Mass., USA) to yield a cDNA. Real-time PCR was conducted using SYBR Green (Life Technologies) for gene expression analysis of immune-inducing factors in the intestines.

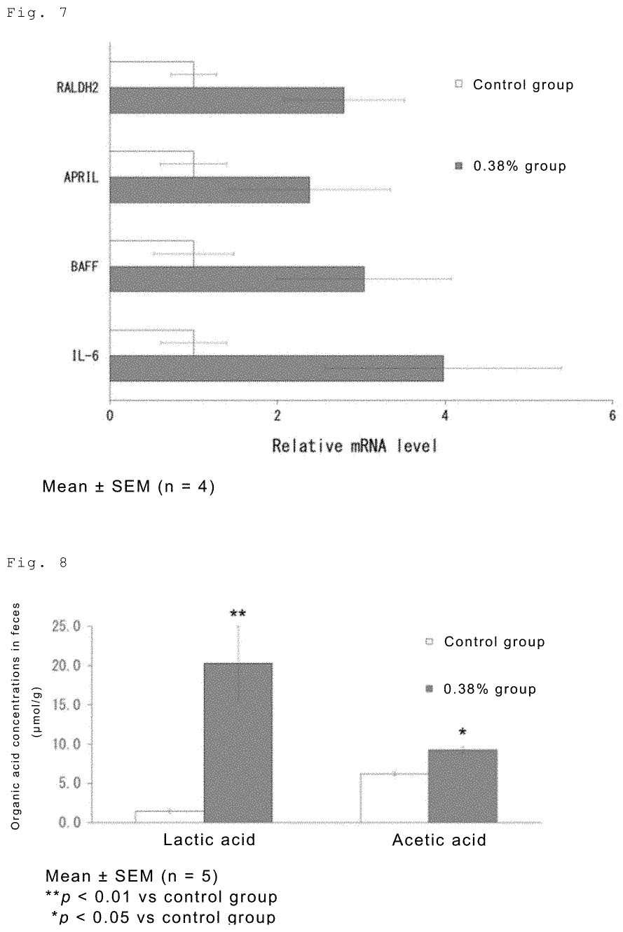

As shown in FIG. 7, the expression levels of the RALDH2, APRIL, BAFF and IL-6 genes in the group fed with the lactic acid bacteria powder were significantly higher or tended to be higher than those in the control group. IL-6, BAFF and APRIL are secreted from dendritic cells upon stimulation and enhance the production of IgA from B cells. Retinoic acid, of which the production is mediated by RALDH2 secreted from dendritic cells, promotes homing of IgA-producing B cells to the mucosa. As apparent from the results shown in FIG. 7, enhancement of IgA levels in the intestinal mucosa by the lactic acid bacteria of the present invention was also observed at the gene expression levels.

The genes analyzed in this Example were retinal dehydrogenase 2 (RALDH2), a proliferation inducing ligand (APRIL), B-cell activating factor (BAFF), and interleukin-6 (IL-6). As an internal control gene, S-actin was used in this Example. The primers used for the gene analysis are shown in Table 7 below.

TABLE 7

| | Forward | Reverse |
|---|---|---|
| RALDH2 | 5′-gacttgtagcagctgtcttcact-3′ | 5′-tcacccatttctctcccatttcc-3′ |
| | (SEQ ID NO: 1) | (SEQ ID NO: 2) |
| APRIL | 5′-tcacaatgggtcaggtggtatc-3′ | 5′-tgtaaatgaaagacacctgcactgt-3′ |
| | (SEQ ID NO: 3) | (SEQ ID NO: 4) |
| BAFF | 5′-tgctatgggtcatgtcatcca-3′ | 5′-ggcagtgttttgggcatattc-3′ |
| | (SEQ ID NO: 5) | (SEQ ID NO: 6) |
| IL-6 | 5′-aatagtccttcctaccccaatttc-3′ | 5′-atttcaagatgaattggatggtct-3′ |
| | (SEQ ID NO: 7) | (SEQ ID NO: 8) |
| β-actin | 5′-catccgtaaagacctctatgccaac-3′ | 5′-atggagccaccgatccaca-3′ |
| (control) | (SEQ ID NO: 9) | (SEQ ID NO: 10) |

### Example 4: Determination of the Amount of Short Chain Fatty Acids or Organic Acids and Measurement of pH

The mice prepared in accordance with the procedures (1) to (4) in Example 1 were fed with the lactic acid bacteria powder. On day 60 after the start of feeding with the lactic acid bacteria powder, the feces of mice excreted per day were collected, lyophilized and suspended in ultrapure water. The suspension of the feces was then left to stand at 4° C. for 1 hour and centrifuged, and the supernatant was filtered. The filtered supernatant was treated with a labeling reagent for short chain fatty acid analysis (YMC CO., LTD., Kyoto, Japan) and the organic acid content (lactic acid, acetic acid, propionic acid and butyric acid) was determined by HPLC. The pH of the filtered supernatant was also measured.

As shown in FIG. 8, the amount of lactic acid and acetic acid in the feces in mice significantly increased in the group fed with the lactic acid bacteria powder as compared with that of the control group (lactic acid: p<0.01, acetic acid: p<0.05).

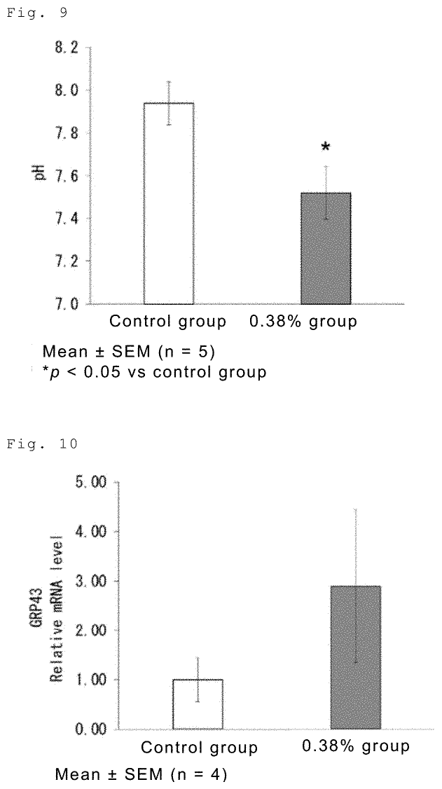

As shown in FIG. 9, the pH of the feces of mice significantly decreased in the group fed with the lactic acid bacteria powder as compared with that of the control group (p<0.05).

### Example 5: Gene Expression Analysis of GRP43

The mice prepared in accordance with the procedures (1) to (4) in Example 1 were fed with the lactic acid bacteria powder. The mice were euthanized by cervical dislocation on day 60 after the start of feeding with the lactic acid bacteria powder, and the Peyer's patch cells were collected from the mice. RNA was extracted using illustra RNAspin Mini RNA Isolation Kit (GE Healthcare, Piscataway, N.J., USA). A 0.15 μg sample of the RNA was weighed and reverse transcribed with oligo primers (Invitrogen, Carlsbad, Calif.) using SuperScript III Reverse Transcriptase (Invitrogen). The reverse transcribed sample was then purified using PCR Purification Kit (Qiagen, Cambridge, Mass., USA) to yield a cDNA. Real-time PCR was conducted using SYBR Green (Life Technologies) for gene expression analysis of GRP43 in the intestines. As an internal control gene, S-actin was used in this Example as with Example 4. The primers used for the gene analysis are shown in Table below.

TABLE 8

| | Forward | Reverse |
|---|---|---|
| GPR43 | 5′-ggcttctacagcagcatcta-3′ | 5′-aagcacaccaggaaattaag-3′ |
| | (SEQ ID NO: 11) | (SEQ ID NO: 12) |

As shown in FIG. 10, the expression level of the GRP43 gene in the group fed with the lactic acid bacteria powder tended to be higher than that in the control group (p=0.42). Statistical analysis was conducted using Excel Statistics software (SSRI, Tokyo, Japan). The data were statistically analyzed by multiple comparison test (Dunnett's test).

### Example 6

Animals, including cattle (dairy cattle, beef cattle), pigs, chickens (broilers, layers), etc., suffering from diarrhea were fed with the lactic acid bacteria or a processed product thereof according to the present invention. The diarrhea stopped, and solidified feces were observed instead of liquid feces. A pharmaceutical product is usually required for raising livestock and poultry, but the amount of such a pharmaceutical product added to feeds for livestock and poultry was reduced, and the incidence of immune diseases (e.g., mastitis, colibacillosis, etc.) was also reduced in cattle and chickens. The mortality rate of broilers was also reduced. The results demonstrate that the lactic acid bacteria of the present invention have immunostimulatory effect.

### Example 7

The lactic acid bacteria or a processed product thereof according to the present invention was administered to a healthy adult human suffering from pollen allergy, and the symptoms were alleviated. The results demonstrate that the lactic acid bacteria of the present invention have antiallergic effect.

### Example 8

The lactic acid bacteria or a processed product thereof according to the present invention was administered to rice, melon, tomato and Chinese cabbage plants. The rice seeds, the melon and tomato fruits and the Chinese cabbage leaves grew larger, indicating that vitality was imparted to these plants. The results demonstrate that the lactic acid bacteria of the present invention have modulating effect on plant growth.

### Example 9

The lactic acid bacteria or a processed product thereof according to the present invention was added to feeds for animals and poultry, and given to cattle (beef cattle), pigs and chickens (broilers). The feed conversion ratio decreased, the physique becomes stronger and other changes were observed as compared with animals and poultry fed with feeds free of the lactic acid bacteria or a processed product thereof. The results demonstrate that the lactic acid bacteria of the present invention have promoting effect on animal growth.

### Example 10

The lactic acid bacteria or a processed product thereof according to the present invention was added to a fertilizer of paddy rice. The immunity of the rice plants was enhanced, making the whole rice plants resistant to lodging as compared with rice plants grown with a fertilizer free of the lactic acid bacteria or a processed product thereof. The results demonstrate that the lactic acid bacteria of the present invention have preventive effect on plant lodging.

### Example 11

The lactic acid bacteria or a processed product thereof according to the present invention was added to a fertilizer of rice. The umami taste of the rice increased as compared with rice grown with a fertilizer free of the lactic acid bacteria or a processed product thereof. The results demonstrate that the lactic acid bacteria of the present invention have adjustment effect on the umami taste of plants.

### Example 12

The nucleotide sequence of the Enterococcus durans bacteria (the E. durans HS-08 strain) of the present invention was determined by a known method and is shown below (SEQ ID NO: 13).

GACGAACGCTGGCGGCGTGCCTAATACATGCAAGTCGTACGGTTCTTTT
TCCACGGGAGCTTGCTCCACCGGAAAAAGAAGAGTGGCGAACGGGTGAG
TAACACGTGGGTAACCTGCCCATCAGAAGGGGATAACACTTGGAAACAG
GTGCTAATACCGTATAACAATCGAAACCGCATGGTTTTGATTTGAAAGG
CGCTTTCGGGTGTCGCTGATGGATGGACCCGCGGTGCATTAGCTAGTTG
GTGAGGTAACGGCTCACCAAGGCCACGATGCATAGCCGACCTGAGAGGG
TGATCGGCCACATTGGGACTGAGACACGGCCCAAACTCCTACGGGAGGC
AGCAGTAGGGAATCTTCGGCAATGGACGAAAGTCTGACCGAGCAACGCC
GCGTGAGTGAAGAAGGTTTTCGGATCGTAAAACTCTGTTGTTAGAGAAG
AACAAGGATGAGAGTAACTGTTCATCCCTTGACGGTATCTAACCAGAAA
GCCACGGCTAACTACGTGCCAGCAGCCGCGGTAATACGTAGGTGGCAAG
CGTTGTCCGGATTTATTGGGCGTAAAGCGAGCGCAGGCGGTTTCTTAAG
TCTGATGTGAAAGCCCCCGGCTCAACCGGGGAGGGTCATTGGAAACTGG
GAGACTTGAGTGCAGAAGAGGAGAGTGGAATTCCATGTGTAGCGGTGAA
ATGCGTAGATATATGGAGGAACACCAGTGGCGAAGGCGGCTCTCTGGTC
TGTAACTGACGCTGAGGCTCGAAAGCGTGGGGAGCAAACAGGATTAGAT
ACCCTGGTAGTCCACGCCGTAAACGATGAGTGCTAAGTGTTGGAGGGTT
TCCGCCCTTCAGTGCTGCAGCTAACGCATTAAGCACTCCGCCTGGGGAG
TACGACCGCAAGGTTGAAACTCAAAGGAATTGACGGGGGCCCGCACAAG
CGGTGGAGCATGTGGTTTAATTCGAAGCAACGCGAAGAACCTTACCAGG
TCTTGACATCCTTTGACCACTCTAGAGATAGAGCTTCCCCTTCGGGGGC
AAAGTGACAGGTGGTGCATGGTTGTCGTCAGCTCGTGTCGTGAGATGTT
GGGTTAAGTCCCGCAACGAGCGCAACCCTTATTGTTAGTTGCCATCATT
CAGTTGGGCACTCTAGCAAGACTGCCGGTGACAAACCGGAGGAAGGTGG
GGATGACGTCAAATCATCATGCCCCTTATGACCTGGGCTACACACGTGC
TACAATGGGAAGTACAACGAGTTGCGAAGTCGCGAGGCTAAGCTAATCT
CTTAAAGCTTCTCTCAGTTCGGATTGCAGGCTGCAACTCGCCTGCATGA
AGCCGGAATCGCTAGTAATCGGCGGATCAGCACGCCGCGGTGAATACGT
TCCCGGGCCTTGTACACACCGCCCGTCACACCACGAGAGTTTGTAACAC
CCGAAGTCGGTGAGGTAACCTTTTTGGAGCCAGCCGCCTAAGGTGGGAT
AGATGATTGGGGTG

The overall results demonstrate that the lactic acid bacteria of the present invention are useful in that, for example, when administered to animals or added to feeds for animals,

(1) the lactic acid bacteria promote the gastrointestinal health of animals (livestock animals) and present infectious diseases etc., and thus reduce the amount of antibiotics etc. used and reduce the mortality rate;
(2) the lactic acid bacteria increase the appetite of animals (livestock animals) and improve the growth promotion rate, and thus shorten the days until the shipment of the animals; and (3) the lactic acid bacteria reduce the odor of the feces of animals (livestock animals) and thus reduce stress from the barn environment and provide good meat quality.

The lactic acid bacteria of the present invention are also useful in that, for example, when sprayed onto plants or added to fertilizers,

(1) the lactic acid bacteria impart vitality to plants, thereby modulating plant growth;
(2) the lactic acid bacteria enhance the immunity of plants, thereby preventing plant lodging; and
(3) the lactic acid bacteria increase the umami taste of plants.

### INDUSTRIAL APPLICABILITY

The present invention provides a novel lactic acid bacterial strain Enterococcus durans (E. durans HS-08 strain) or a processed product thereof, and a method for increasing the number of the bacteria or the amount of the processed product, all of which are industrially applicable.

1. A Enterococcus durans HS-08 bacterium (Accession No. NITE BP-02675) or a processed product thereof.

2. A composition for induction of IgA production or for immunostimulation, the composition comprising the bacterium or a processed product thereof according to claim 1.

3. An antiallergic composition comprising the bacterium or a processed product thereof according to claim 1.

4. A composition for inhibition of proliferation of harmful bacteria and/or pathogenic bacteria, the composition comprising the bacterium or a processed product thereof according to claim 1.

5. A composition for protection of mucosa, the composition comprising the bacterium or a processed product thereof according to claim 1.

6. A composition for at least one application selected from the group consisting of enhancement of production of short chain fatty acids, enhancement of production of organic acids, and upregulation of gene expression of GRP43, the composition comprising the bacterium or a processed product thereof according to claim 1.

7. A composition for at least one application selected from the group consisting of modulation of plant growth, prevention of plant lodging, and adjustment of umami taste of a plant, the composition comprising the bacterium or a processed product thereof according to claim 1.

8. A composition for promotion of growth of an animal and/or for enhancement of immunity in an animal, the composition comprising the bacterium or a processed product thereof according to claim 1.

9. A fertilizer for plants, comprising the bacterium or a processed product thereof according to claim 1.

10. A feed for animals, comprising the bacterium or a processed product thereof according to claim 1.

11. A food or drink product, a cosmetic product or a pharmaceutical product, comprising the bacterium or a processed product thereof according to claim 1.

12. A concrete member comprising the bacterium or a processed product thereof according to claim 1.

13. A composition comprising the bacterium or a processed product thereof according to claim 1 and at least one selected from the group consisting of beneficial bacteria including other lactic acid bacteria and bifidobacteria, beneficial yeasts, and molds, or a processed product thereof.

14. A method for increasing the number of Enterococcus durans HS-08 bacteria, the method comprising culturing Enterococcus durans HS-08 bacteria (Accession No. NITE BP-02675).

15. A method for induction of IgA production or for immunostimulation in a subject comprising administering an effective amount of the composition comprising the bacterium or a processed product thereof according to claim 1 to said subject.

16. A method of treating allergies in a subject comprising administering an antiallergic composition comprising administering an effective amount of the bacterium or a processed product thereof according to claim 1 to said subject.

17. A method for inhibition of proliferation of harmful bacteria and/or pathogenic bacteria in a subject comprising administering an effective amount of the composition comprising the bacterium or a processed product thereof according to claim 1 to said subject.

18. A method for protection of mucosa in a subject comprising administering an effective amount of the composition comprising the bacterium or a processed product thereof according to claim 1 to said subject.

19. A method for enhancing production of short chain fatty acids, enhancing production of organic acids, and/or upregulating gene expression of GRP43 in a subject comprising administering an effective amount of the composition comprising the bacterium or a processed product thereof according to claim 1 to said subject.

20. A method for modulating plant growth, preventing plant lodging, and/or adjusting the umami taste of a plant comprising administering an effective amount of the composition comprising the bacterium or a processed product thereof according to claim 1 to a plant.

21. A method for promoting growth and/or enhancing immunity of an animal comprising administering the composition comprising administering an effective amount of the bacterium or a processed product thereof according to claim 1 to said animal.


SEQUENCE LISTING (13 sequences)

SEQ ID NO: 1 - DNA, 23 bp, Artificial Sequence
  primer
gacttgtagcagctgtcttcact

SEQ ID NO: 2 - DNA, 23 bp, Artificial Sequence
  primer
tcacccatttctctcccatttcc

SEQ ID NO: 3 - DNA, 22 bp, Artificial Sequence
  primer
tcacaatgggtcaggtggtatc

SEQ ID NO: 4 - DNA, 25 bp, Artificial Sequence
  primer
tgtaaatgaaagacacctgcactgt

SEQ ID NO: 5 - DNA, 21 bp, Artificial Sequence
  primer
tgctatgggtcatgtcatcca

SEQ ID NO: 6 - DNA, 21 bp, Artificial Sequence
  primer
ggcagtgttttgggcatattc

SEQ ID NO: 7 - DNA, 24 bp, Artificial Sequence
  primer
aatagtccttcctaccccaatttc

SEQ ID NO: 8 - DNA, 24 bp, Artificial Sequence
  primer
atttcaagatgaattggatggtct

SEQ ID NO: 9 - DNA, 25 bp, Artificial Sequence
  primer
catccgtaaagacctctatgccaac

SEQ ID NO: 10 - DNA, 19 bp, Artificial Sequence
  primer
atggagccaccgatccaca

SEQ ID NO: 11 - DNA, 20 bp, Artificial Sequence
  primer
ggcttctacagcagcatcta

SEQ ID NO: 12 - DNA, 20 bp, Artificial Sequence
  primer
aagcacaccaggaaattaag

SEQ ID NO: 13 - DNA, 1483 bp, Enterococcus durans
gacgaacgctggcggcgtgcctaatacatgcaagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtg